(19) United States
(12) Patent Application Publication
(10) Pub. No.: US 2023/0335267 A1
(43) Pub. Date: Oct. 19, 2023

(54) FORMULATION INFORMATION MANAGEMENT DEVICE, METHOD, AND COMPUTER-READABLE MEDIUM

(71) Applicant: KANEKA CORPORATION, Osaka (JP)

(72) Inventor: Hirokazu Sakamoto, Takasago-shi (JP)

(73) Assignee: KANEKA CORPORATION, Osaka (JP)

(21) Appl. No.: 18/213,542

(22) Filed: Jun. 23, 2023

Related U.S. Application Data
(63) Continuation of application No. PCT/JP2021/036425, filed on Oct. 1, 2021.

(30) Foreign Application Priority Data
Dec. 25, 2020 (JP) 2020-216364

(51) Int. Cl. G16H 40/20; G16H 50/80

(52) U.S. Cl. CPC G16H 40/20; G16H 50/80; G16H 10/40

(57) ABSTRACT

A formulation information management method includes: acquiring, by a server from a first terminal, individual information associated with a first individual and infection information indicating an infectious disease with which the first individual has been infected, as results of examining infectious diseases of livestock; acquiring, by the server from a second terminal, the individual information and formulation information indicating a blood product manufactured from blood containing blood collected when dissecting the first individual; generating, by the server, related information that associates the infection information and the formulation information based on the individual information; storing, by the server, the related information in a storage; and acquiring, by the server, infection information of a second individual; and in response to acquiring the infection information of the second individual, outputting, by the server, the formulation information associated by the related information with infection history information including the infection information.

### CROSS-REFERENCE TO RELATED APPLICATIONS

This application claims priority to Japanese Patent Application No. 2020-216364, filed Dec. 25, 2020, the entire content of which is incorporated herein by reference.

### TECHNICAL FIELD

The present invention relates to a formulation information management device, a formulation information management method, and a computer-readable medium.

### BACKGROUND ART

In the related art, various types of systems for managing information about raw materials in a product manufacturing process and providing the information to processors of the raw materials have been proposed.

For example, technology for collecting information about a product and providing the collected information to consumers in order to enable them to select products with peace of mind is disclosed in Patent Document 1. In this technology, information about raw materials obtained from a raw material provider, information about a processed product from a raw material processor, and information about a product recipient from the product recipient are acquired as information to be provided to consumers. A product provider is also capable of obtaining information about the product to be provided to the recipient in this technology.

### CITATION LIST

### Patent Document

### Patent Document 1

Japanese Patent No. 6621953

### Technical Problem

Meanwhile, there are formulation manufacturers who manufacture formulations (hereinafter referred to as “animal blood-based formulations”) using animal blood (an example of a raw material) among formulation manufacturers (examples of processors of raw materials). The characteristics of animal blood may affect the efficacy of some animal blood-based formulations. The characteristics of animal blood can differ according to an individual animal whose blood is used in a manufacturing process. For example, a breeding method based on an individual difference between animals, a breeding environment, given feed, a medical history, and the like can have an influence on the characteristics of animal blood. Therefore, some animal blood-based formulations may have a more effective effect on a treatment target animal if they are manufactured using the blood of animals bred in a method closer to a method in which the treatment target animal has been bred. Therefore, it is desirable for providers of animal blood-based formulations to be able to provide animal blood-based formulations in consideration of a method of breeding a treatment target animal. However, in the technology described in Patent Document 1, it is difficult to acquire information about animal blood-based formulations in consideration of a method of breeding a treatment target animal.

In view of the above-described problems of conventional technology, one or more embodiments of the present invention provide a formulation information management device, a formulation information management method, and a computer-readable medium capable of providing an animal blood-based formulation more effective for animal treatment.

### Solution to Problem

### SUMMARY

In order to achieve a solution to the aforementioned problems, in one aspect of the present invention, a formulation information management method according to one or more embodiments includes: acquiring, by a server from a first terminal, individual information associated with a first individual and infection information indicating an infectious disease with which the first individual has been infected, as results of examining infectious diseases of livestock; acquiring, by the server from a second terminal, the individual information and formulation information indicating a blood product manufactured from blood containing blood collected when dissecting the first individual; generating, by the server, related information that associates the infection information and the formulation information based on the individual information; storing, by the server, the related information in a storage; and acquiring, by the server, infection information of a second individual; and in response to acquiring the infection information of the second individual, outputting, by the server, the formulation information associated by the related information with infection history information including the input infection information.

In other embodiments, a formulation information management device may include: a storage unit configured to store formulation information about a formulation manufactured using blood of an animal as a raw material and animal information about the animal having the blood in association; an input information acquisition unit configured to acquire input information input from a terminal of an output destination of output information via a network; an output information acquisition unit configured to acquire at least one item within the formulation information corresponding to the input information acquired by the input information acquisition unit and the animal information about the animal having the blood as the output information from the storage unit; and an output processing unit configured to output the output information acquired by the output information acquisition unit to the terminal of the output destination via the network.

In another aspect of the present invention, a formulation information management device according to one or more embodiments includes: a controller that: acquires, from a first terminal, individual information associated with a first individual and infection information indicating an infectious disease with which the first individual has been infected, as results of examining infectious diseases of livestock; acquires, from a second terminal, the individual information and formulation information indicating a blood product manufactured from blood containing blood collected when dissecting the first individual; and a storage that: generates related information that associates the infection information and the formulation information based on the individual information, and stores the related information, wherein the controller receives infection information of a second individual, and in response to acquiring the infection information of the second individual, the controller outputs the formulation information associated by the related information with infection history information including the input infection information.

In other embodiments, a formulation information management method including: a process in which a storage unit stores formulation information about a formulation manufactured using blood of an animal as a raw material and animal information about the animal having the blood in association; a process in which an input information acquisition unit acquires input information input from a terminal of an output destination of output information via a network; a process in which an output information acquisition unit acquires at least one item within the formulation information corresponding to the input information acquired by the input information acquisition unit and the animal information about the animal having the blood as the output information from the storage unit; and a process in which an output processing unit outputs the output information acquired by the output information acquisition unit to the terminal of the output destination via the network.

In yet another aspect of the present invention, a non-transitory computer-readable medium (CRM) according to one or more embodiments causes a formulation information management device to: acquire, from a first terminal, individual information associated with a first individual and infection information indicating an infectious disease with which the first individual has been infected, as results of examining infectious diseases of livestock; acquire, from a second terminal, the individual information and formulation information indicating a blood product manufactured from blood containing blood collected when dissecting the first individual; generate related information that associates the infection information and the formulation information based on the individual information; store the related information; acquire infection information of a second individual; and in response to acquiring the infection information of the second individual, output the formulation information associated by the related information with infection history information including the input infection information.

In other embodiments, a CRM may cause a computer to function as: a storage means configured to store formulation information about a formulation manufactured using blood of an animal as a raw material and animal information about the animal having the blood in association; an input information acquisition means configured to acquire input information input from a terminal of an output destination of output information via a network; an output information acquisition means configured to acquire at least one item within the formulation information corresponding to the input information acquired by the input information acquisition means and the animal information about the animal having the blood as the output information from the storage means; and an output processing means configured to output the output information acquired by the output information acquisition means to the terminal of the output destination via the network.

### Technological Improvements

One or more embodiments of the present invention provide an improved technology for manufacturing more effective animal blood-based formulations for animal treatment.

### BRIEF DESCRIPTION OF DRAWINGS

FIG. 1 is a diagram showing an outline of an information processing system according to one or more embodiments.

FIG. 2 is a block diagram showing an example of a functional configuration of an information management server according to one or more embodiments.

FIG. 3 is a diagram showing an example of related information according to one or more embodiments.

FIG. 4 is a diagram showing an example of individual information according to one or more embodiments.

FIG. 5 is a diagram showing an example of breeding information according to one or more embodiments.

FIG. 6 is a diagram showing an example of blood information according to one or more embodiments.

FIG. 7 is a diagram showing an example of formulation information according to one or more embodiments.

FIG. 8 is a flowchart showing a flow of a process of an information management server according to one or more embodiments.

### DETAILED DESCRIPTION

Hereinafter, embodiments of the present invention will be described with reference to the drawings.

### <1. System Overview>

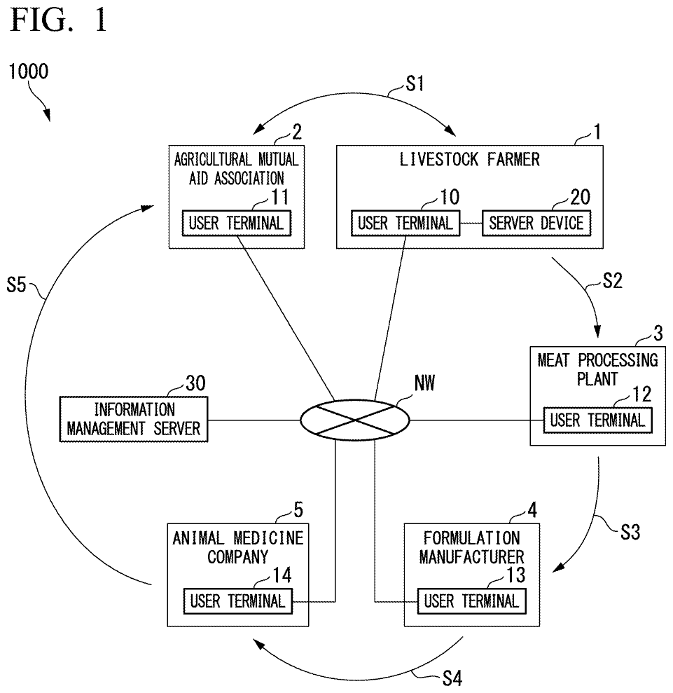

First, an overview of an information processing system according to one or more embodiments will be described with reference to FIG. 1. FIG. 1 is a diagram showing an overview of the information processing system according to one or more embodiments. As shown in FIG. 1, an information processing system 1000 includes user terminals 10 to 14, a sensor device (“sensor”) 20, and an information management server 30.

The user terminals 10 to 14 are examples of terminals that can be operated by a user who is a livestock farmer 1, a user of an agricultural mutual aid association 2, a user of a meat processing plant 3, a user of a formulation manufacturer 4, and a user of an animal medicine company 5. The user terminals 10 to 14 are, for example, terminals such as personal computers (PCs), smartphones, and tablet terminals. Also, the user terminals 10 to 14 may be operable only at places where they are installed or may be portable and operable at any location.

The user terminals 10 to 14 are communicatively connected with the information management server 30 via a network NW. The user terminals 10 to 14 can transmit information input to the user to the information management server 30 or output information received from the information management server 30.

The livestock farmer 1 is a livestock breeder. The livestock farmer 1 breeds, for example, animals such as cows, pigs, chickens, horses, and sheep as livestock. The type of livestock bred by the livestock farmer 1 is not limited to these examples. Hereinafter, in one or more embodiments, an example in which the livestock farmer 1 breeds an animal for meat as livestock will be described. In one or more embodiments, the livestock farmer 1 delivers the animal bred as livestock to the meat processing plant 3.

The breeder (an example of the user who is the livestock farmer 1) can input information of an animal bred as livestock (hereinafter also referred to as “animal information”) to the user terminal 10. Animal information includes, for example, information about an individual animal (hereinafter also referred to as “individual information”) and information about breeding of an animal (hereinafter also referred to as “breeding information”). Also, information included in the animal information is not limited to this example. For example, it may include area information indicating an area where the livestock has been raised. Details of information included in the individual information and the breeding information will be described below. The input animal information is transmitted to the information management server 30 via the network NW and stored in the information management server 30.

Also, the breeder can operate the user terminal 10 to refer to information stored in the information management server 30.

The agricultural mutual aid association 2 is a corporation operated by farmers as members. The agricultural mutual aid association 2 is an example of an organization to which veterinarians who treat livestock belong and the organization is not limited to the agricultural mutual aid association. As mentioned above, the veterinarians belong to the agricultural mutual aid association 2. The veterinarians examine and treat animals bred by the livestock farmer 1 as an example. The veterinarians are not limited to veterinarians belonging to the agricultural mutual aid association 2. For example, a veterinarian may be a veterinarian who operates his or her own veterinary clinic.

The veterinarian (an example of a user of the agricultural mutual aid association 2) can input information about medical examination and treatment to the user terminal 11. The information about the medical examination is, for example, information indicating a disease name, symptoms, and the like obtained in the medical examination. The information about the treatment is, for example, information indicating a name of a prescribed drug, a name of treatment such as surgery, and the like. The information about the input medical examination or treatment is transmitted to the information management server 30 via the network NW and stored in the information management server 30. The information about the medical examination and treatment is stored in the information management server 30 and hence managed as a history. In the history, a history of the information about the medical examination is hereinafter also referred to as “medical history information.”

Also, the veterinarian can operate the user terminal 11 to refer to information stored in the information management server 30.

The meat processing plant 3 is a place where animals are dissected and processed into meat. The meat processing plant 3 may also be a slaughterhouse where the animals are slaughtered. A meat processing operator collects blood from the animal when it is dissected in the meat processing plant 3. In one or more embodiments, the meat processing operator draws or exsanguinates blood of animals purchased from the livestock farmer 1 and collects the blood at the meat processing plant 3. Also, the meat processing operator sells (provides) the collected (acquired) blood of the animals to the formulation manufacturer 4. The meat processing operator may collect blood in a blood collection process before the animal dissection work or may collect blood through exsanguination in the dissection work.

The meat processing operator (an example of a user of the meat processing plant 3) can input information about blood collected from the dissected animal (hereinafter also referred to as “blood information”) to the user terminal 12. Details of the information included in the blood information will be described below. The input blood information is transmitted to the information management server 30 via the network NW and stored in the information management server 30.

Also, the meat processing operator can operate the user terminal 12 to refer to information stored in the information management server 30.

The formulation manufacturer 4 is a manufacturer that manufactures a formulation made from the blood of an animal (hereinafter also referred to as “animal blood-based formulation”) and is an example of a manufacturer. The animal is an animal dissected in the meat processing plant 3 and the blood is blood provided from the meat processing plant 3.

In the animal blood-based formulations, the characteristics of the animal blood used in a manufacturing process may or may not affect the efficacy. Hereinafter, in one or more embodiments, an example in which an animal blood-based formulation is a formulation whose efficacy can change with the characteristics of the animal blood used in the manufacturing process will be described. For example, the characteristics of the animal blood may differ according to the individual animal whose blood is used in the manufacturing process. Specifically, a breeding method based on an individual difference between animals, a breeding environment, given feed, a medical history, and the like can have an influence on the characteristics of animal blood. In one or more embodiments, information indicating a breeding method that can affect changes in the characteristics of animal blood is included in animal information. In the information indicating the breeding method, information indicating an individual difference between animals is included in individual information as an example and information indicating the breeding environment, the given feed, the medical history, and the like is included in the breeding information as an example. That is, the animal blood-based formulation according to one or more embodiments is a formulation indicating characteristics according to the animal information.

Examples of animal blood-based formulations according to one or more embodiments include a globulin formulation. Globulin formulations are drugs purified from antibodies in the blood and used as a therapeutic medicine for infectious diseases. Thus, the efficacy of the globulin formulation is affected by a type of antibody contained in the blood used in the manufacturing process and the type of antibody contained in the blood is affected by a history of infection (an example of the breeding method) with infectious diseases of the animal having the blood.

In one or more embodiments, the formulation manufacturer 4 manufactures an animal blood-based formulation using blood purchased from the meat processing plant 3, and sells the manufactured animal blood-based formulation (example of “blood product”) to the animal medicine company 5. A sales destination of animal blood-based formulations from the formulation manufacturer 4 is not limited to the animal medicine company 5.

The user of the formulation manufacturer 4 can input information about the manufactured animal blood-based formulation (hereinafter also referred to as “formulation information”) to the user terminal 13. Details of the information included in the formulation information will be described below. The input formulation information is transmitted to the information management server 30 via the network NW and stored in the information management server 30.

The user of the formulation manufacturer 4 can operate the user terminal 13 to refer to information stored in the information management server 30.

The animal medicine company 5 is a company that sells animal medicines. In one or more embodiments, the animal medicine company 5 sells animal blood-based formulations purchased from the formulation manufacturer 4 to veterinarians belonging to the agricultural mutual aid association 2. The sales destination of animal blood-based formulations from the animal medicine company 5 is not limited to veterinarians belonging to the agricultural mutual aid association 2.

The user of the animal medicine company 5 can operate the user terminal 14 to refer to information stored in the information management server 30.

Here, relationships between the livestock farmer 1, the agricultural mutual aid association 2, the meat processing plant 3, the formulation manufacturer 4, and the animal medicine company 5 in one or more embodiments will be described in a series of flows.

First, the livestock farmer 1 breeds animals as livestock for meat. The livestock may be bred inside of a barn or by grazing outside of the barn. The livestock farmer 1 may request a veterinarian belonging to the agricultural mutual aid association 2 to examine and treat the animals that are bred (step S1). The veterinarian of the agricultural mutual aid association 2 examines and treats the animals when there is a request from the livestock farmer 1.

When it is time to deliver the animals being bred, the livestock farmer 1 delivers the animals to the meat processing plant 3 (step S2).

At the meat processing plant 3, animals purchased from the livestock farmer 1 are dissected and processed so that they can be delivered as meat. At the meat processing plant 3, the blood of the animals is collected before and after the dissection. The collected blood is sold from the meat processing plant 3 to the formulation manufacturer 4 (step S3).

The formulation manufacturer 4 manufactures animal blood-based formulations using the blood purchased from the meat processing plant 3. The formulation manufacturer 4 sells the manufactured animal blood-based formulations to the animal medicine company (step S4).

The animal medicine company 5 sells animal blood-based formulations purchased from the formulation manufacturer 4 to the veterinarians of the agricultural mutual aid association 2 (step S5). The veterinarians can use animal blood-based formulations purchased from the animal medicine company 5 for the treatment of animals being bred as livestock by the livestock farmer 1.

The sensor device 20 acquires information about the livestock breeding environment and information about the livestock being bred. The sensor device 20 is provided, for example, in the barn of the livestock farmer 1. A position where the sensor device 20 is provided may be either inside or outside of the barn. Also, the sensor device 20 may be attached to livestock as a wearable terminal as an example. Also, the types and number of sensor devices 20 are not particularly limited.

The sensor device 20 is communicatively connected with the user terminal 10 through wired communication or wireless communication. The information management server 30 acquires information from the sensor device 20, which may be done either directly from the sensor device 20 or indirectly through the user terminal 10. The sensor device 20 can transmit the acquired information to the user terminal 10. The user terminal 10 can transmit information received from the sensor device 20 to the information management server 30 via the network NW.

The sensor device 20 may be communicatively connected with the information management server 30 via the network NW. In this case, the sensor device 20 can directly transmit the acquired information to the information management server 30 without involving the user terminal 10.

The sensor device 20 acquires, for example, information such as images (still images/moving images) and sounds indicating temperatures and humidities inside and outside of the barn and states inside and outside of the barn, as information about the livestock breeding environment. Also, the sensor device 20 acquires images (still images/moving images) and sounds indicating a state of livestock or acquires information about an amount of eaten feed, an amount of drunk water, a body temperature, a heart rate, a respiratory rate, a blood oxygen saturation level, milking, childbirth, reproduction, estrus, a sexual cycle, and other various types of behavior (recumbent, standing, defecation, urination, eating, exercise, rumination, and the like) as information about the livestock being bred.

Also, the information acquired by the sensor device 20 is not limited to this example. Also, the information acquired by the sensor device 20 may be included in the breeding information.

The information management server 30 is a server that manages various types of information and is an example of a formulation information management device. For example, the information management server 30 stores information input to the user terminals 10 to 14, information acquired by the sensor device 20, and the like in association. Specifically, the information management server 30 stores information in which animal information (including at least breeding information) and formulation information are at least associated (hereinafter also referred to as “related information”). In one or more embodiments, an example of related information in which animal information, blood information, and formulation information are associated will be described. Specifically, the related information is information associated with animal information, blood information of blood collected from the animal indicated in the animal information, and formulation information of an animal blood-based formulation manufactured using the blood. Details of the information included in the related information will be described below.

Also, the information management server 30 acquires information corresponding to information input from the user terminals 10 to 14 from the stored information and outputs the acquired information to the user terminals 10 to 14.

Hereinafter, in one or more embodiments, as an example, an example in which the information management server 30 generates and stores related information, animal information (individual information and breeding information), blood information, and formulation information on the basis of information input from the user terminal 10 of the livestock farmer 1, the user terminal 11 of the agricultural mutual aid association 2, the user terminal 12 of the meat processing plant 3, and the user terminal 13 of the formulation manufacturer 4 will be described. Hereinafter, in one or more embodiments, as an example, an example in which the information management server 30 acquires information corresponding to the information input from the user terminal 14 of the animal medicine company 5 from the stored information and outputs the acquired information to the user terminal 14 (an output destination terminal) of the animal medicine company 5 will be described.

### <2. Configuration of Information Management Server>

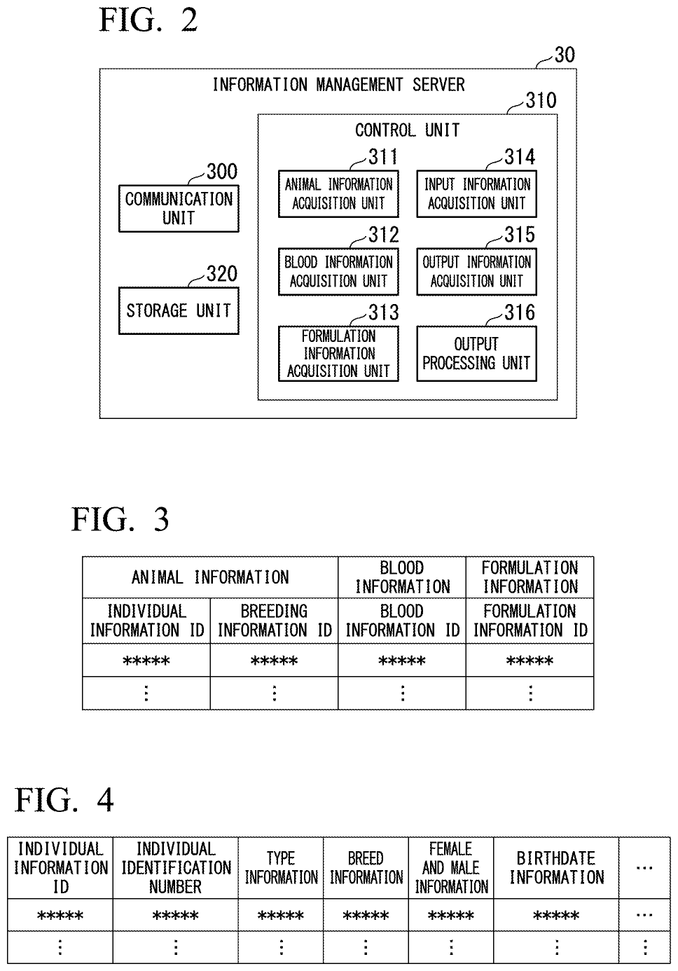

An overview of the information processing system 1000 according to one or more embodiments has been described above. Subsequently, a functional configuration of the information management server 30 according to one or more embodiments will be described with reference to FIGS. 2 to 7. FIG. 2 is a block diagram showing an example of a functional configuration of the information management server 30 according to one or more embodiments. As shown in FIG. 2, the information management server 30 includes a communication unit 300, a control unit (“controller”) 310, and a storage unit (“storage”) 320.

### (1) Communication Unit 300

The communication unit 300 has a function of performing various types of communication. For example, the communication unit 300 is connected with a network NW using wired local area network (LAN) communication or the like and performs various types of communication via the network NW. Also, the communication unit 300 is connected with the user terminals 10 to 14, for example, via the network NW, and performs various types of communication with the user terminals 10 to 14. The communication unit 300 may be connected to the sensor device 20 via the network NW and perform various types of communication with the sensor device 20.

### (2) Control Unit 310

The control unit 310 has a function of controlling the entire operation of the information management server 30. For example, the control unit 310 is implemented when the information management server 30 causes a CPU provided as hardware to execute instructions as an example.

As shown in FIG. 2, the control unit 310 includes an animal information acquisition unit 311, a blood information acquisition unit 312, a formulation information acquisition unit 313, an input information acquisition unit 314, an output information acquisition unit 315, and an output processing unit 316.

### (2-1) Animal Information Acquisition Unit 311

The animal information acquisition unit 311 has a function of acquiring animal information. For example, the animal information acquisition unit 311 acquires animal information input from the user terminal via the network NW through communication with the communication unit 300. The animal information acquisition unit 311 causes the storage unit 320 to store the acquired animal information.

As an example, in one or more embodiments, because individual information is input from the user terminal 10 of the livestock farmer 1, the animal information acquisition unit 311 acquires individual information input from the user terminal 10 of the livestock farmer 1 and causes the storage unit 320 to store the acquired individual information. Also, in one or more embodiments, because breeding information is input from the user terminal 10 of the livestock farmer 1 or the user terminal 11 of the agricultural mutual aid association 2, the animal information acquisition unit 311 acquires the breeding information input from the user terminal 10 of the livestock farmer 1 or the user terminal 11 of the agricultural mutual aid association 2 and causes the storage unit 320 to store the acquired breeding information.

### (2-2) Blood Information Acquisition Unit 312

The blood information acquisition unit 312 has a function of acquiring blood information. For example, the blood information acquisition unit 312 acquires blood information input from the user terminal via the network NW through communication with the communication unit 300. The blood information acquisition unit 312 causes the storage unit 320 to store the acquired blood information.

As an example, in one or more embodiments, because the blood information is input from the user terminal 12 of the meat processing plant 3, the blood information acquisition unit 312 acquires the blood information input from the user terminal 12 of the meat processing plant 3 and causes the storage unit 320 to store the acquired blood information.

### (2-3) Formulation Information Acquisition Unit 313

The formulation information acquisition unit 313 has a function of acquiring formulation information. For example, the formulation information acquisition unit 313 acquires formulation information input from the user terminal via the network NW through communication with the communication unit 300. The formulation information acquisition unit 313 causes the storage unit 320 to store the acquired formulation information.

As an example, in one or more embodiments, because the formulation information is input from the user terminal 13 of the formulation manufacturer 4, the formulation information acquisition unit 313 acquires the formulation information input from the user terminal 13 of the formulation manufacturer 4 and causes the storage unit 320 to store the acquired formulation information.

### (2-4) Input Information Acquisition Unit 314

The input information acquisition unit 314 has a function of acquiring input information. The input information is information input to the user terminal when the user refers to information stored in the information management server 30. For example, the input information acquisition unit 314 acquires the input information input from the user terminal via the network NW through communication with the communication unit 300. As an example, in one or more embodiments, because the input information is input from the user terminal 14 of the animal medicine company 5, the input information acquisition unit 314 acquires input information input from the user terminal 14 of the animal medicine company 5.

The input information is, for example, at least one of animal information about a treatment target animal (including but not limited to individual information associated with the target animal, infection information indicating an infectious disease, and symptom information about symptoms of the treatment target animal) and formulation information. The input information is not limited to this example. For example, animal information about animals other than the treatment target animal and information about symptoms of the animals may be input as the input information.

### (2-5) Output Information Acquisition Unit 315

The output information acquisition unit 315 has a function of acquiring output information. The output information is information output to a user terminal that has input the input information when the user refers to information stored in the information management server 30. The output information acquisition unit 315 acquires the output information from the information stored in the storage unit 320 on the basis of the input information acquired by the input information acquisition unit 314. Specifically, the output information acquisition unit 315 acquires at least one of animal information and formulation information corresponding to the input information as the output information from the storage unit 320. Also, the output information acquisition unit 315 may acquire blood information as the output information from the storage unit 320.

More specifically, when the input information acquired by the input information acquisition unit 314 is animal information about the treatment target animal, the output information acquisition unit 315 acquires formulation information corresponding to the animal information acquired by the input information acquisition unit 314 as the output information from the storage unit 320. Specifically, the output information acquisition unit 315 acquires related information including animal information identical to the input animal information from the storage unit 320 and outputs the formulation information included in the acquired related information as the output information.

According to this configuration, the user can acquire formulation information corresponding to the animal information on the basis of the animal information (including information indicating a breeding method) of the treatment target animal. That is, the user can examine animal blood-based formulations that can act more effectively on the treatment target animal from the viewpoint of the breeding method of the treatment target animal.

Also, when the input information acquired by the input information acquisition unit 314 is symptom information of the treatment target animal, the output information acquisition unit 315 acquires formulation information corresponding to the symptom information acquired by the input information acquisition unit 314 as output information from the storage unit 320. Specifically, the output information acquisition unit 315 acquires related information including symptom information identical to the input symptom information from the storage unit 320 and outputs the formulation information included in the acquired related information as the output information. In one or more embodiments, the symptom information is included in the medical history information, the medical history information is included in the breeding information, and the breeding information is included in the animal information. That is, when related information including symptom information identical to the input symptom information is acquired, the output information acquisition unit 315 refers to animal information included in the related information stored in the storage unit 320.

According to this configuration, the user can examine animal blood-based formulations that can more effectively act on the treatment target animal from the viewpoint of the symptoms of the treatment target animal.

Also, when the input information acquired by the input information acquisition unit 314 is formulation information, the output information acquisition unit 315 acquires animal information about an animal having blood corresponding to the formulation information acquired by the input information acquisition unit 314 as output information from the storage unit 320. Specifically, the output information acquisition unit 315 acquires related information including formulation information identical to the input formulation information from the storage unit 320 and outputs animal information included in the acquired related information as the output information.

According to this configuration, the user can examine animals suitable for using animal blood-based formulations corresponding to the formulation information from the viewpoint of the formulation information.

As described above, the user (for example, the animal medicine company 5) can acquire information about an animal blood-based formulation corresponding to the animal by inputting information about the treatment target animal. Also, the user can acquire information about the animal corresponding to the animal blood-based formulation by inputting information about the animal blood-based formulation. The user can examine an animal blood-based formulation effective for the treatment target animal or examine an animal suitable as the target for using the animal blood-based formulation on the basis of the acquired information. The user can select an animal blood-based formulation that acts more effectively on the treatment target animal on the basis of an examination result. Consequently, the user can provide animal blood-based formulations more effective for animal treatment.

### (2-6) Output Processing Unit 316

The output processing unit 316 has a function of controlling the output of output information. For example, the output processing unit 316 transmits the output information acquired by the output information acquisition unit 315 to the user terminal via the network NW through communication with the communication unit 300 and causes the output information to be output. As an example, in one or more embodiments, because input information is input from the user terminal 14 of the animal medicine company 5, the output processing unit 316 transmits the output information to the user terminal 14 of the animal medicine company 5 and causes the output information to be output.

### (3) Storage Unit 320

The storage unit 320 includes a storage medium provided as hardware in the information management server 30, for example, a hard disk drive (HDD), a solid-state drive (SSD), a flash memory, an electrically erasable programmable read only memory (EEPROM), a random access read/write memory (RAM), a read only memory (ROM), or any combination of these storage media.

The storage unit 320 has a function of storing various types of information. For example, the storage unit 320 stores related information, animal information (individual information and breeding information), blood information, and formulation information.

FIG. 3 is a diagram showing an example of the related information according to one or more embodiments. As shown in FIG. 3, the related information includes, for example, an individual information identification (ID), a breeding information ID, a blood information ID, and a formulation information ID. The individual information ID and the breeding information ID are classified as information related to animal information, the blood information ID is classified as information related to blood information, and the formulation information ID is classified as information related to formulation information. Also, the information included in the related information is not limited to this example.

The individual information ID is information for identifying individual information and any value is set uniquely. The breeding information ID is information for identifying the breeding information and any value is set uniquely. The blood information ID is information for identifying blood information and any value is set uniquely. The formulation information ID is information for identifying the formulation information and any value is set uniquely.

FIG. 4 is a diagram showing an example of the individual information according to one or more embodiments. As shown in FIG. 4, the individual information includes, for example, information indicating an individual information ID, an individual identification number, type information, breed information, female and male information, birthdate information, and the like. In the individual information shown in FIG. 4, for example, the type information, the breed information, the female and male information, and the birthdate information are information indicating an individual difference and are examples of information indicating a breeding method. Also, the information included in the individual information is not limited to this example.

The individual information ID is information for identifying individual information and any value is set uniquely. The individual information ID corresponds to the individual information ID included in the related information. The individual identification number is information for identifying an individual animal and any value is set uniquely. The type information is information indicating a type of animal. The breed information is information indicating a breed of an animal. The female and male information is information indicating whether an animal is male or female. The birthdate information is information indicating a birthdate of an animal.

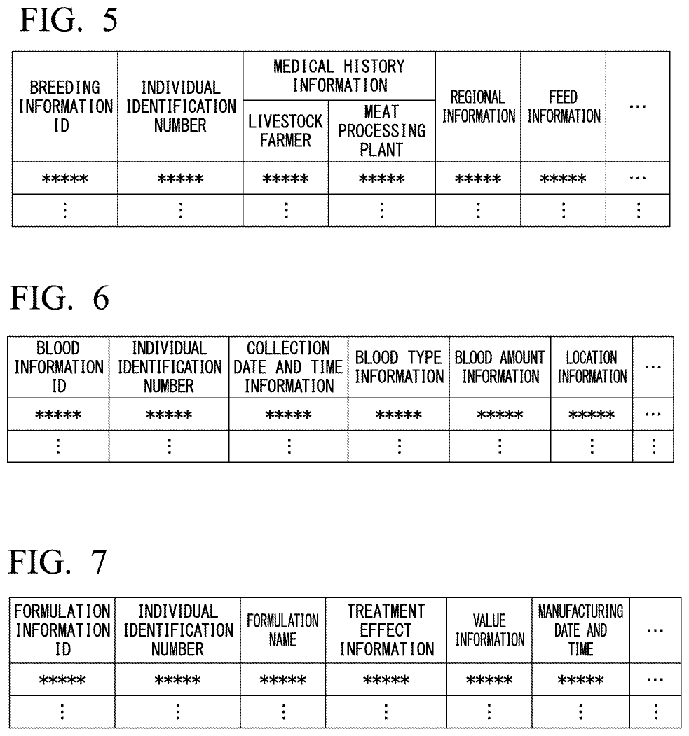

FIG. 5 is a diagram showing an example of the breeding information according to one or more embodiments. As shown in FIG. 5, the breeding information includes, for example, information indicating a breeding information ID, an individual identification number, medical history information (the livestock farmer and the meat processing plant), regional information, feed information, and the like. In the breeding information shown in FIG. 5, for example, medical history information (the livestock farmer and the meat processing plant), regional information, and feed information are examples of information indicating a breeding method. Also, information included in the breeding information is not limited to this example.

The breeding information ID is information for identifying breeding information and any value is set uniquely. The breeding information ID corresponds to the breeding information ID included in the related information. The individual identification number is information for identifying an individual animal and any value is set uniquely. The individual identification number corresponds to the individual identification number included in the individual information. The medical history information is information indicating a medical history of an animal. Specifically, the medical history information includes a history of diseases found when animals were bred by the livestock farmer 1 (the livestock farmer in the table) and a history of diseases found at the meat processing plant 3 (the meat processing plant in the table). The history of diseases includes, for example, information indicating a disease name and symptoms (symptom information). At the meat processing plant 3, animal diseases can be found in an examination process performed before the dissection and in an analysis or examination process during the dissection or after the dissection. That is, the history of diseases found at the meat processing plant 3 includes diseases found in at least one of a stage before the animal dissection, a stage during the animal dissection, and a stage after the animal dissection. More specifically, the medical history information is infection history information indicating an infection history of infectious diseases of the animal. The medical history information may include results of examining viruses and microorganisms such as bacteria, viruses, fungi, protozoa, and mycoplasma. Viruses and microorganisms are examined, for example, on the blood of animals and the examination results show viruses and microorganisms contained in the blood of animals. The viruses and microorganisms may be examined when the animal is bred by the livestock farmer 1 or at any timing before, during, or after the dissection of the animal at the meat processing plant 3. Depending on the type of virus or microorganism in the blood, the characteristics of an animal's blood can change. That is, the type of virus/microorganism contained in the blood can also affect the efficacy of animal blood-based formulations. The regional information is information indicating a region where the animals were bred. The feed information is information about feed and feed additives used in animal breeding.

The medical history information is included in the breeding information of the animal information and therefore the user can examine animal blood-based formulations effective for a disease (for example, an infectious disease) of the treatment target animal. Also, the regional information is included in the breeding information of the animal information and therefore the user can examine the effective animal blood-based formulation in accordance with the region where the treatment target animal was bred. Also, the feed information is included in the breeding information of the animal information and therefore the user can examine the effective animal blood-based formulation in accordance with feed given to the treatment target animal.

For example, when the user inputs infection information such as any one of the infectious disease names and symptoms of the treatment target animal, the region name and address where the animal was bred, the feed name or feed additive name given to the animal, and the like as input information, the output information acquisition unit 315 acquires the formulation information associated with the breeding information (animal information) including the input information from the storage unit 320. Also, the formulation information acquired by the output information acquisition unit 315 is output to the user terminal by the output processing unit 316.

Also, the medical history information, the regional information, the feed information, and the like are included in the breeding information of the animal information and therefore the user can examine information about individuals for which an animal blood-based formulation is effective.

For example, when a user inputs the formulation information of an animal blood-based formulation as the input information, the output information acquisition unit 315 acquires animal information associated with the formulation information including the input information from the storage unit 320. Also, the animal information acquired by the output information acquisition unit 315 is output to the user terminal by the output processing unit 316.

FIG. 6 is a diagram showing an example of the blood information according to one or more embodiments. As shown in FIG. 6, the blood information includes, for example, information indicating a blood information ID, an individual identification number, collection date and time information, blood type information, blood amount information, blood volume information indicating an amount of the blood, location information, and the like. Also, the information included in the blood information is not limited to this example.

The blood information ID is information for identifying blood information and any value is set uniquely. The blood information ID corresponds to the blood information ID included in the related information. The individual identification number is information for identifying individual blood and any value is set uniquely. The collection date and time information is information indicating the date and time when the blood was collected. The blood type information is information indicating a blood type of blood. The blood amount information is information indicating a blood amount. The location information is information indicating a location where the blood was collected (the meat processing plant).

FIG. 7 is a diagram showing an example of the formulation information according to one or more embodiments. As shown in FIG. 7, the formulation information includes, for example, information indicating a formulation information ID, an individual identification number, a formulation name, treatment effect information, value information, a manufacturing date and time, and the like. Also, the information included in the formulation information is not limited to this example. For example, the formulation information may include therapeutic effect information indicating therapeutic effect of the blood product and manufacturing date or time information that indicates a date and time when the blood product has been manufactured.

The formulation information ID is information for identifying the formulation information and any value is set uniquely. The formulation information ID corresponds to the formulation information ID included in the related information. The individual identification number is information for identifying an individual animal blood-based formulation and any value is set uniquely. The formulation name is information indicating a name of the animal blood-based formulation. The treatment effect information is information indicating the treatment effect of the animal blood-based formulation. The treatment effect information is set on the basis of, for example, a usage record of the animal blood-based formulation. The value information is information indicating a meat value of an animal treated with the animal blood-based formulation. Specifically, the value information includes a grade of the meat rating of the individual to which the animal blood-based formulation was administered, a price set on the basis of the grade, an impression by a consumer who ate the meat (for example, regarding taste), and the like. The manufacturing date and time is information indicating a date and time when the animal blood-based formulation was manufactured.

### <3. Processing Flow>

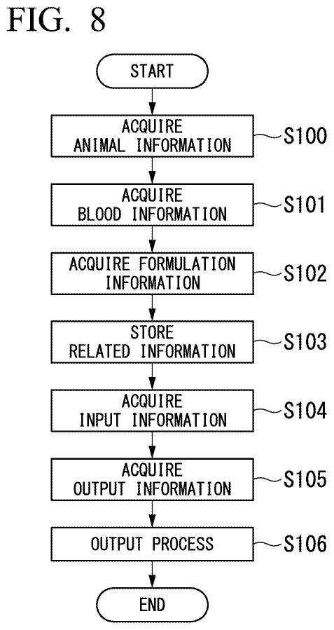

A functional configuration of the information management server 30 according to one or more embodiments has been described above. Subsequently, a flow of a process of the information management server 30 according to one or more embodiments will be described with reference to FIG. 8. FIG. 8 is a flowchart showing the flow of the process of the information management server 30 according to one or more embodiments.

First, when animal information is input by the user terminal, the animal information acquisition unit 311 of the information management server 30 acquires the animal information via the network NW through communication with the communication unit 300 (step S100). As an example, in one or more embodiments, because the animal information is input from the user terminal 10 of the livestock farmer 1 or the user terminal 11 of the agricultural mutual aid association 2, the animal information acquisition unit 311 acquires the animal information from the user terminal 10 of the livestock farmer 1 or the user terminal 11 of the agricultural mutual aid association 2 and causes the storage unit 320 of the information management server 30 to store the acquired animal information.

Subsequently, when blood information has been input by the user terminal, the blood information acquisition unit 312 of the information management server 30 acquires the blood information via the network NW through communication with the communication unit 300 (step S101). As an example, in one or more embodiments, because the blood information has been input from the user terminal 12 of the meat processing plant 3, the blood information acquisition unit 312 acquires the blood information from the user terminal 12 of the meat processing plant 3 and causes the storage unit 320 to store the acquired blood information.

Subsequently, when formulation information has been input by the user terminal, the formulation information acquisition unit 313 of the information management server acquires the formulation information via the network NW through communication with the communication unit 300 (step S102). As an example, in one or more embodiments, because the formulation information is input from the user terminal 13 of the formulation manufacturer 4, the formulation information acquisition unit 313 acquires the formulation information from the user terminal 13 of the formulation manufacturer 4 and causes the storage unit 320 to store the acquired formulation information.

Subsequently, when animal information, blood information, and formulation information have been stored, the storage unit 320 stores related information in which the animal information, the blood information, and the formulation information are associated (step S103).

Subsequently, when input information has been input by the user terminal, the input information acquisition unit 314 of the information management server 30 acquires the input information via the network NW through communication with the communication unit 300 (step S104). As an example, in one or more embodiments, because the input information is input from the user terminal 14 of the animal medicine company 5, the input information acquisition unit 314 acquires the input information from the user terminal 14 of the animal medicine company 5.

Subsequently, when the input information has been acquired by the input information acquisition unit 314, the output information acquisition unit 315 of the information management server 30 acquires output information from the information stored in the storage unit 320 on the basis of the input information (step S105).

Also, when the output information has been acquired by the output information acquisition unit 315, the output processing unit 316 of the information management server 30 transmits the output information to the user terminal via the network NW through communication with the communication unit 300 and causes the output information to be output (step S106). As an example, in one or more embodiments, because the input information is input from the user terminal 14 of the animal medicine company 5, the output processing unit 316 transmits the output information to the user terminal 14 of the animal medicine company 5 and causes the output information to be output.

As described above, the information management server 30 according to one or more embodiments includes the storage unit 320, the input information acquisition unit 314, the output information acquisition unit 315, and the output processing unit 316. The storage unit 320 stores formulation information about a formulation manufactured using animal blood as a raw material and animal information about an animal having the blood in association. The input information acquisition unit 314 acquires input information input from the user terminal to which the output information is output via the network NW. The output information acquisition unit 315 acquires at least one of the formulation information and animal information corresponding to the input information acquired by the input information acquisition unit 314 as output information from the storage unit. The output processing unit 316 outputs the output information acquired by the output information acquisition unit 315 to the output destination user terminal via the network NW.

According to this configuration, when input information about the treatment target animal has been input from the user terminal, the information management server 30 according to one or more embodiments outputs information about animal blood-based formulations corresponding to the animal to the user terminal. Specifically, the information management server 30 acquires formulation information associated with animal information of an animal having blood used in a process of manufacturing an animal blood-based formulation corresponding to the animal information of the treatment target animal input and outputs the acquired formulation information to the user terminal. Thereby, the user (for example, the animal medicine company 5) can examine animal blood-based formulations effective for the treatment target animal on the basis of the acquired information. Also, when input information about animal blood-based formulations has been input from the user terminal, the information management server outputs information about the animal corresponding to the animal blood-based formulation to the user terminal. Specifically, the information management server 30 acquires the input formulation information and animal information associated with the corresponding formulation information and outputs the acquired information to the user terminal. Thereby, the user (for example, the animal medicine company 5) can examine an appropriate animal as a target for using animal blood-based formulations on the basis of the acquired information. Thereby, the user can select an animal blood-based formulation that acts more effectively on the treatment target animal on the basis of an examination result.

Consequently, the information management server 30 according to one or more embodiments enables animal blood-based formulations more effective for animal treatment to be provided.

Above, embodiments of the present invention have been described.

Although an example in which the livestock delivered from the livestock farmer 1 to the meat processing plant is used for meat has been described in the above-described embodiments, the present invention is not limited to this example. For example, the livestock delivered from the livestock farmer 1 to the meat processing plant may be livestock bred for the purpose of obtaining milk, dairy products, eggs, and the like. Livestock bred for this purpose can be delivered to the meat processing plant 3 when it is disused as an example. Disused livestock is processed as raw materials such as pet food as an example.

Although an example in which the formulation manufacturer 4 is a manufacturer that manufactures animal blood-based formulations and the animal medicine company 5 is a company that sells the animal blood-based formulations to doctors belonging to the agricultural mutual aid association 2 has been described in the above-described embodiments, the present invention is not limited to this example. For example, the formulation manufacturer 4 may directly sell the animal blood-based formulations to a doctor belonging to the agricultural mutual aid association 2 without involving the animal medicine company 5. That is, the role of the formulation manufacturer 4 and the role of the animal medicine company 5 in the above-described embodiments may be implemented by one company.

Although a specific example in which the animal blood-based formulation is a globulin formulation in which the characteristics of the animal blood used in a manufacturing process affect the efficacy has been described in the above-described embodiments, the present invention is not limited to this example. The animal blood-based formulation whose efficacy is affected by the characteristics of the animal blood used in the manufacturing process may be a blood-based formulation other than the globulin formulation.

Also, the animal blood-based formulation may be one in which the characteristics of the animal blood used in the manufacturing process do not affect the efficacy. Animal blood-based formulations in which the characteristics of animal blood used in the manufacturing process do not affect efficacy are, for example, albumin formulations. Albumin formulations are formulations administered to replenish albumin in the blood when the concentration of albumin in treatment target blood has been decreased. Also, animal blood-based formulations in which the characteristics of animal blood used in the manufacturing process do not affect the efficacy are not limited to such examples and may be blood-based formulations other than albumin formulations.

Although an example in which the output information acquisition unit 315 acquires animal information about an animal having blood corresponding to the formulation information as the output information from the storage unit 320 when the input information acquired by the input information acquisition unit 314 is formulation information has been described in the above-described embodiments, the present invention is not limited to this example. For example, when the input information acquired by the input information acquisition unit 314 is formulation information, the output information acquisition unit 315 may acquire formulation information corresponding to the formulation information acquired by the input information acquisition unit 314 as output information from the storage unit 320. As an example, when the individual identification number of the animal blood-based formulation has been acquired as the input information, the output information acquisition unit 315 acquires formulation information including the individual identification number from the storage unit 320. Also, the formulation information acquired by the output information acquisition unit 315 is output to the user terminal by the output processing unit 316.

According to this configuration, the user (for example, the animal medicine company 5) can examine treatment effect information, value information, and the like based on the usage record of the animal blood-based formulation. The user can analyze animal blood-based formulations on the basis of the examined treatment effect information, value information, and the like. Also, the user can add an additional value according to an analysis result to the animal blood-based formulation and set a price of the animal blood-based formulation according to the added value. For example, the user can analyze the characteristics of animal blood-based formulations on the basis of the treatment effect information and assign an additional value and set a price according to the characteristics. Also, the user can analyze a relationship between animal blood-based formulations and meat on the basis of the value information and assign an additional value and set a price according to the relationship.

Also, some or all of components provided in the information management server 30 in the above-described embodiments may be implemented by a computer. In this case, these functions may be implemented by recording instructions for implementing the control functions on a computer-readable medium and causing a computer system to read and execute the instructions recorded on the recording medium. Also, it is assumed that the “computer system” used herein includes an operating system (OS) and hardware such as peripheral equipment. Also, the “computer-readable medium” refers to a flexible disk, a magneto-optical disc, a ROM, a portable medium such as a compact disc (CD)-ROM, or a storage such as a hard disk embedded in the computer system. Further, the “computer-readable medium” may include a computer-readable medium for dynamically holding the instructions for a short time period as in a communication line when the instructions are transmitted via a network such as the Internet or a communication circuit such as a telephone circuit and a computer-readable medium for holding the instructions for a given time period as in a volatile memory inside the computer system serving as a server or a client when the instructions are transmitted. Also, the above-described instructions may be a program for implementing some of the above-described functions. Further, the above-described instructions may be a program capable of implementing the above-described function in combination with a program already recorded on the computer system or may be implemented using a programmable logic device such as a field programmable gate array (FPGA).

Although embodiments of the present invention have been described in detail above with reference to the drawings, specific configurations are not limited to the embodiments and various design changes and the like may also be made without departing from the scope of the present invention.

### REFERENCE SIGNS LIST

1 Livestock farmer
2 Agricultural mutual aid association
3 Meat processing plant
4 Formulation manufacturer
5 Animal medicine company
10 to 14 User terminal
Sensor device
30 Information management server
300 Communication unit
310 Control unit
311 Animal information acquisition unit
312 Blood information acquisition unit
313 Formulation information acquisition unit
314 Input information acquisition unit
315 Output information acquisition unit
316 Output processing unit
320 Storage unit
NW Network

What is claimed is:

1. A formulation information management method comprising:
acquiring, by a server from a first terminal, individual information associated with a first individual and infection information indicating an infectious disease with which the first individual has been infected, as results of examining infectious diseases of livestock;
acquiring, by the server from a second terminal, the individual information and formulation information indicating a blood product manufactured from blood containing blood collected when dissecting the first individual;
generating, by the server, related information that associates the infection information and the formulation information based on the individual information;
storing, by the server, the related information in a storage; and
acquiring, by the server, infection information of a second individual; and
in response to acquiring the infection information of the second individual, outputting, by the server, the formulation information associated by the related information with infection history information including the infection information.

2. The formulation information management method according to claim 1, further comprising:
acquiring, by the server from a third terminal, blood information indicating the blood collected when dissecting the first individual and the individual information associated with the first individual;
generating, by the server, related information that associates infection history information of the first individual with the blood information based on the individual information,
storing, by the server, the related information in the storage; and
in response to acquiring the infection information of the second individual, outputting, by the server, the blood information associated by the related information with the infection history information including the infection information.

3. The formulation information management method according to claim 1, further comprising:
acquiring, by the server from a third terminal, the individual information and area information indicating an area where the livestock has been raised;
generating, by the server, related information that associates the area information and the formulation information based on the individual information,
storing, by the server, the related information in the storage; and
in response to acquiring the area information, outputting, by the server, the formulation information associated with the area information by the related information.

4. The formulation information management method according to claim 1, further comprising:
acquiring, by the server from a sensor attached to the first individual, another information of the first individual;
acquiring, by the server from a third terminal, the individual information associated with the first individual and the another information of the first individual;
generating, by the server, related information that associates the another information of the first individual and the formulation information based on the individual information,
storing, by the server, the related information in the storage; and
in response to acquiring the formulation information, outputting, by the server, the another information of the first individual associated with the formulation information by the related information.

5. The formulation information management method according to claim 1, further comprising:
acquiring, by the server from a sensor installed in a livestock barn, another information of the first individual;
acquiring, by the server from a third terminal, the individual information associated with the first individual;
generating, by the server, related information that associates the another information of the first individual and the formulation information based on the individual information,
storing, by the server, the related information in the storage; and
in response to acquiring the formulation information, outputting, by the server, the another information of the first individual associated with the formulation information by the related information.

6. The formulation information management method according to claim 1, further comprising:
acquiring, by the server from a third terminal, information indicating a slaughterhouse where the first individual is slaughtered and the individual information associated with the first individual;
generating, by the server, related information that associates the information indicating the slaughterhouse and the formulation information based on the individual information;
storing, by the server, the related information in the storage; and
in response to acquiring the formulation information, outputting, by the server, the information indicating the slaughterhouse associated with the formulation information by the related information.

7. The formulation information management method according to claim 1, wherein the formulation information includes therapeutic effect information indicates a therapeutic effect of the blood product and manufacturing date or time information that indicates a date and time when the blood product has been manufactured.

8. The formulation information management method according to claim 2, wherein the blood information includes collection date and time information indicating a date and time when the blood has been collected, blood type information indicating a blood type of the blood, blood volume information indicating an amount of the blood, and location information indicating a place where the blood has been collected.

9. A formulation information management device comprising:
a controller that:
acquires, from a first terminal, individual information associated with a first individual and infection information indicating an infectious disease with which the first individual has been infected, as results of examining infectious diseases of livestock;
acquires, from a second terminal, the individual information and formulation information indicating a blood product manufactured from blood containing blood collected when dissecting the first individual; and
a storage that:
generates related information that associates the infection information and the formulation information based on the individual information, and
stores the related information, wherein
the controller receives infection information of a second individual, and
in response to acquiring the infection information of the second individual, the controller outputs the formulation information associated by the related information with infection history information including the infection information.

10. A non-transitory computer-readable medium (CRM) for causing a formulation information management device to:
acquire, from a first terminal, individual information associated with a first individual and infection information indicating an infectious disease with which the first individual has been infected, as results of examining infectious diseases of livestock;
acquire, from a second terminal, the individual information and formulation information indicating a blood product manufactured from blood containing blood collected when dissecting the first individual;
generate related information that associates the infection information and the formulation information based on the individual information;
store the related information;
acquire infection information of a second individual; and
in response to acquiring the infection information of the second individual, output the formulation information associated by the related information with infection history information including the infection information.
